(19) United States
(12) Patent Application Publication
(10) Pub. No.: US 2025/0213230 A1
(43) Pub. Date: Jul. 3, 2025

(54) METHOD FOR PERFORMING ULTRASONIC SCANNING ON BREAST AND AN ULTRASONIC IMAGING SYSTEM

(71) Applicant: GE Precision Healthcare LLC, Waukesha, WI (US)

(72) Inventors: Chao Zhou, Wuxi (CN); Xin Wang, Wuxi (CN); Liye Pei, Wuxi (CN)

(21) Appl. No.: 18/990,890

(22) Filed: Dec. 20, 2024

(30) Foreign Application Priority Data
Dec. 29, 2023 (CN) 202311863988.X

(51) Int. Cl. A61B 8/00; A61B 8/08

(52) U.S. Cl. CPC A61B 8/54; A61B 8/0825

(57) ABSTRACT

A method for performing ultrasonic scanning on a breast, and an ultrasonic imaging system. The ultrasonic scanning is at least partially performed by means of a scanning assembly, the scanning assembly comprising a frame, a scanning probe and a driving apparatus being accommodated in the frame, and the driving apparatus driving the scanning probe to move within the frame to perform the ultrasonic scanning. The method includes: performing pre-scanning of the breast, to generate a pre-scanned ultrasonic image of the breast; performing image recognition on the pre-scanned ultrasonic image, to obtain the size of a mammary gland of the breast; and on the basis of the size of the mammary gland, performing scan planning on the scanning assembly, and using the scanning assembly to perform scan imaging of the breast on the basis of the scan planning.

### CROSS-REFERENCE TO RELATED APPLICATION

This application claim priority to Chinese Patent Application No. 202311863988.X, which was file on Dec. 29, 2023 at the Chinese Patent Office. The entire contents of the above-listed application are incorporated by reference herein in their entirety.

### TECHNICAL FIELD

The present application relates to the field of medical imaging and relates in particular to a method for performing ultrasonic scanning on a breast and an ultrasonic imaging system.

### BACKGROUND

Ultrasonic imaging is one of the important means for imaging the interior of the body of a person to be scanned. Generally, ultrasonic imaging systems use ultrasonic transducers to convert electrical energy into ultrasonic pulses. The ultrasonic pulses are sent to the interior of the body of the person to be scanned, and echo signals are generated. The echo signals are received by transducer elements, and are then converted to electrical signals. The electrical signals are processed by a specialized processing device to form a desired ultrasonic image.

Ultrasonic imaging systems have important applications in scanning many organs of the body. For example, a full-field breast ultrasonic scanning device may be used to image breast tissue in one or a plurality of planes. During full-field breast ultrasonic scanning, it is often necessary to perform multiple scans on a single breast and then determine whether the breast region is completely covered or not, and if not, it is necessary to increase a scanning region. The above process consumes a lot of time.

It should be noted that the above introduction of the background is only for the convenience of clearly and completely describing the technical solutions of the present application, and for the convenience of understanding for those skilled in the art. The above technical solutions are not considered to be well known to those skilled in the art merely because they are set forth in the Background of the present application.

### SUMMARY

The inventors have found that, in practice, standardized procedures for full-field breast ultrasonic scanning may result in redundancy of the scanning workflow for different individual subjects. For example, the coverage of a scanning assembly of a full-field breast ultrasonic scanning device is different for different sizes of mammary glands. For example, a medium-sized mammary gland may have most of its area covered by the scanning assembly, while a small-sized mammary gland may be completely covered by the scanning assembly. However, the size of the mammary gland hidden under the skin is difficult to estimate directly. Following standard scanning procedure is still the current mainstream of full-field breast ultrasonic scanning.

In order to solve at least one of the above problems or other similar problems, embodiments of the present application provide a method for performing ultrasonic scanning on a breast, and an ultrasonic imaging system, wherein the size of a mammary gland is determined by pre-scanning the breast before a main scan, scan planning is performed on the scanning assembly on the basis of the size of the mammary gland, and scan imaging of the breast is performed on the basis of the scan planning. This can save scanning time and improve a scanning success rate.

According to one aspect of an embodiment of the present application, a method for performing ultrasonic scanning on a breast is provided; the ultrasonic scanning is at least partially performed by a scanning assembly, the scanning assembly comprising a frame, a scanning probe and a driving apparatus being accommodated in the frame, and the driving apparatus driving the scanning probe to move within the frame to perform the ultrasonic scanning, and the method for ultrasonic scanning comprises:
Performing pre-scanning of the breast, to generate a pre-scanned ultrasonic image of the breast;
Performing image recognition on the pre-scanned ultrasonic image, to obtain the size of a mammary gland of the breast; and
On the basis of the size of the mammary gland, performing scan planning on the scanning assembly, and using the scanning assembly to perform scan imaging of the breast on the basis of the scan planning.

In some embodiments, pre-scanning the breast comprises:
Acquiring ultrasonic data about the breast in at least one angular direction, the ultrasonic data comprising at least ultrasonic data of a nipple edge of the breast and ultrasonic data extending outward along the nipple edge to an edge of the mammary gland.

In some embodiments, a probe used for performing the pre-scanning is an additional probe or the scanning probe, wherein the additional probe comprises at least one of a 2D probe and a volumetric ultrasonic probe.

In some embodiments, the at least one angular direction comprises a plurality of angular directions, the plurality of angular directions being uniformly distributed in a circumferential direction.

In some embodiments, performing image recognition on the pre-scanned ultrasonic image to obtain the size of the mammary gland of the breast comprises:

Recognizing the mammary gland in the pre-scanned ultrasonic image, determining an outer edge of the mammary gland, and obtaining the size of the mammary gland of the breast on the basis of the outer edge of the mammary gland.

In some embodiments, the method further comprises:
Performing image recognition on the pre-scanned ultrasonic image, to obtain a chest wall in the pre-scanned ultrasonic image, and determining a depth of the scan imaging on the basis of the chest wall.

In some embodiments, the depth of the scan imaging is a maximum value among chest wall depths in the pre-scanned ultrasonic image.

In some embodiments, the scan planning comprises at least one of a number of scans, a scanning position, and a scanning stroke of the scanning assembly.

In some embodiments, the method further comprises:
Displaying the scan planning on a display.

In some embodiments, the method further comprises:
Monitoring a scanning process of the scanning assembly in real time during the scan imaging;
Matching the scanning process and the scan planning; and
Displaying a matching result.

In some embodiments, the scan planning comprises:
In single-scan imaging, planning so that the driving apparatus does not drive the scanning probe to move outside the mammary gland; and/or
In multi-scan imaging, planning so that the driving apparatus does not drive the scanning probe to scan an overlapping region between a plurality of scans.

According to a second aspect of the embodiments of the present application, an ultrasonic imaging system is provided, the system comprising:
A scanning assembly moving over the surface of a breast to acquire ultrasonic echo signals; and
A processor configured to execute the method according to any one of the foregoing embodiments.

According to yet another aspect of the embodiments of the present application, a non-transitory computer-readable medium having a computer program stored thereon is provided, the computer program having at least one code segment, and the at least one code segment being executable by a machine so that the machine executes steps of the method according to any one of the foregoing embodiments.

One of the beneficial effects of the embodiments of the present application is that, by means of determining the size of the mammary gland by pre-scanning the breast before a main scan, scan planning is performed on the scanning assembly on the basis of the size of the mammary gland, and scan imaging of the breast is performed on the basis of the scan planning. This can save scanning time and improve a scanning success rate.

With reference to the following description and drawings, specific implementations of the present application are disclosed in detail. It should be understood that the implementations of the present application are not limited in scope thereby. Within the scope of the spirit and clauses of the appended claims, the embodiments of the present application include many changes, modifications, and equivalents.

The features described and/or illustrated for one implementation may be used in one or more other implementations in the same or similar manner, be combined with features in other embodiments, or replace features in other implementations.

It should be emphasized that the terms “include/comprise/have”, when used herein, refer to the presence of features, integrated components, or assemblies, but do not preclude the presence or addition of one or more other features, integrated components, or assemblies.

### BRIEF DESCRIPTION OF THE DRAWINGS

The above and other objects, features and advantages of the embodiments of the present application will become more apparent from the following detailed description with reference to the drawings, in which:

FIG. 1 is a perspective view of an ultrasonic imaging system according to an embodiment of the present application;

FIG. 2 is a block diagram of the ultrasonic imaging system according to an embodiment of the present application;

FIG. 3 is a perspective view of a scanning assembly of the ultrasonic imaging device according to an embodiment of the present application;

FIG. 4 is a schematic diagram of a method for performing ultrasonic scanning on a breast according to an embodiment of the present application;

FIG. 5 is a schematic diagram of pre-scanning a breast according to a method in an embodiment of the present application;

FIG. 6 is a schematic diagram of a pre-scanned ultrasonic image obtained after pre-scanning according to the method of FIG. 5;

FIG. 7 is a schematic diagram of the size of a mammary gland;

FIG. 8 is a schematic diagram of a chest wall in a pre-scanned ultrasonic image;

FIG. 9 is a schematic diagram of scan planning corresponding to different sizes of mammary glands;

FIG. 10 is a schematic diagram of matching a scanning process and scan planning; and

FIG. 11 is a schematic diagram of displaying scan planning on a display.

### DETAILED DESCRIPTION

The foregoing and other features of the embodiments of the present application will become apparent from the following description with reference to the drawings. In the description and drawings, specific implementations of the present application are disclosed in detail, and some implementations in which the principles of the present application may be employed are indicated. It should be understood that the present application is not limited to the described implementations, and includes all modifications, variations, and equivalents which fall within the scope of the appended claims.

In the embodiments of the present application, the terms “first”, “second”, “upper”, “lower”, etc. are used to distinguish different elements with respect to naming, but do not represent a spatial arrangement, a temporal order, or the like of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar manner, be combined with features in other embodiments, or replace features in other implementations.

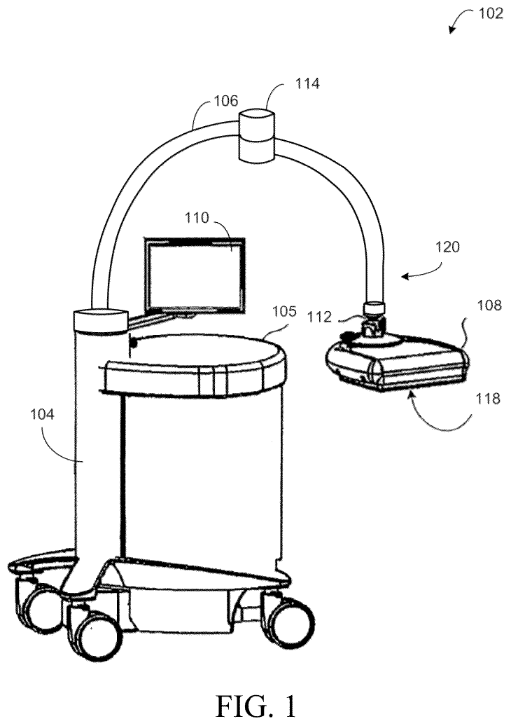

FIG. 1 shows a perspective view of an ultrasonic imaging device 102 according to some embodiments. The body of the ultrasonic imaging system 102 may include a main device, a display 110, an adjustable arm 106, and a scanning assembly 108. Wherein the main device may include a body frame 104, an ultrasonic processor housing 105, and an ultrasonic processor inside the housing 105. The specific structure of each component will be illustrated in detail below.

The body frame 104, the ultrasonic processor housing 105 containing the ultrasonic processor, a movable and adjustable support arm (for example, an adjustable arm) 106 including a hinge joint 114, the scanning assembly 108 connected to a first end 120 of the adjustable arm 106 by means of a ball and socket connector (for example, a ball joint) 112, and the display 110 connected to the body frame 104. The display 110 is connected to the body frame 104 at a joining point where the adjustable arm 106 enters the body frame 104. Since the display 110 is directly connected to the body frame 104 rather than the adjustable arm 106, the display 110 does not affect the weight of the adjustable arm 106 and a balancing mechanism of the adjustable arm 106. In one example, the display 110 is rotatable in horizontal and lateral directions (for example, rotatable around a central axis of the body frame 104), but is not vertically movable. In an alternative example, the display 110 may also be vertically movable. Although FIG. 1 depicts the display 110 connected to the body frame 104, in other examples, the display 110 may be connected to different components of the imaging system 102, such as, connected to the ultrasonic processor housing 105, or positioned away from the imaging system 102.

In one embodiment, the adjustable arm 106 is configured and adapted such that the pressing/scanning assembly 108 (i) is neutrally buoyant in space, or (ii) has a light net downward weight (for example, 1-2 kg) for pressing the breast, while allowing easy user operation. In an alternative embodiment, the adjustable arm 106 is configured such that the scanning assembly 108 is neutrally buoyant in space during positioning of a scanner on tissue of a patient. Then, after the scanning assembly 108 is positioned, internal components of the imaging system 102 may be adjusted to apply a desired downward weight for pressing the breast and improving image quality. In one example, the downward weight (for example, a force) may be in the range of 2-11 kg.

As described above, the adjustable arm 106 includes the hinge joint 114. The hinge joint 114 divides the adjustable arm 106 into a first arm portion and a second arm portion. The first arm portion is connected to the scanning assembly 108 and the second arm portion is connected to the body frame 104. The hinge joint 114 allows the second arm portion to rotate relative to the second arm portion and the body frame 104. For example, the hinge joint 114 allows the scanning assembly 108 to translate transversely and horizontally, but not vertically, relative to the second arm portion and the body frame 104. In such manner, the scanning assembly 108 can rotate toward the body frame 104 or away from the body frame 104. However, the hinge joint 114 is configured to allow the entire adjustable arm 106 (for example, the first arm portion and the second arm portion) to move vertically together as a whole (for example, translating upward and downward along with the body frame 104).

The scanning assembly 108 may include a film assembly 118 having a film that is in a substantially tensioned state to be at least partially attached, for pressing the breast. The film assembly 118 has a bottom surface for contacting the breast, and when the bottom surface is in contact with the breast, the transducer sweeps over a top surface of the film to scan the breast. In one example, the film is a tensioned fabric sheet.

The film assembly 118 may further include an outer frame and a film. The film is fixedly disposed in the outer frame, and the outer frame is detachably connected to the scanning assembly. In an ultrasonic imaging process performed by the ultrasonic imaging system, one side surface of the film can be at least partially in contact with an ultrasonic transducer, and another side surface of the film at least partially in contact with a tissue to be scanned. Such an arrangement can ensure that the ultrasonic transducer transmits and receives signals with less attenuation, and can fix the breast to be scanned to facilitate scanning.

Optionally, the adjustable arm may include a potentiometer (not shown) to allow position and direction sensing performed by the pressing/scanning assembly 108, or may use other types of position and direction sensing (such as gyroscope, magnetic, optical, and radio frequency (RF)). A fully functional ultrasonic engine may be provided within the ultrasonic processor housing 105, and is configured to drive the ultrasonic transducer, and generate volumetric breast ultrasound data from a scan in conjunction with related position and orientation information. In some examples, volumetric scan data may be transmitted to another computer system by using any of a variety of data transmission methods known in the art so as to be further processed, or the volumetric scan data may be processed by the ultrasonic engine. A general-purpose computer/processor integrated with the ultrasonic engine may further be provided for general user interface and system control. The general-purpose computer may be a self-contained stand-alone unit, or may be remotely controlled, configured, and/or monitored by remote stations connected across networks.

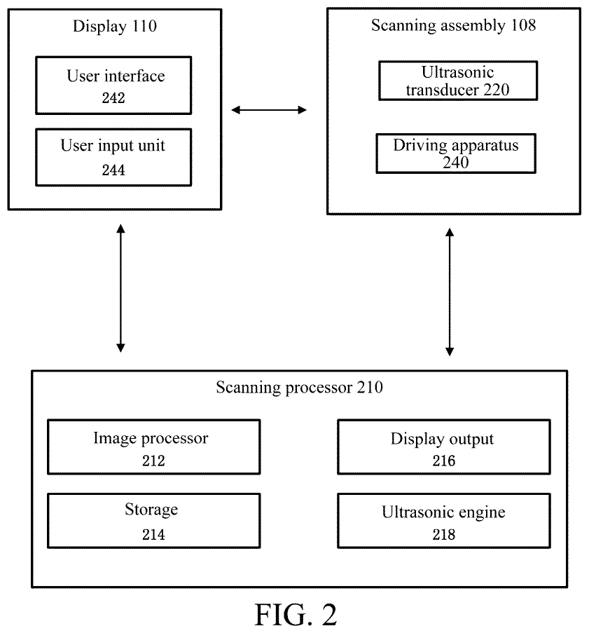

FIG. 2 is a block diagram 200 that schematically illustrates various system parts of the ultrasonic imaging system 102, including the scanning assembly 108, the display 110, and a scanning processor 210. In one example, the scanning processor 210 may be included within the ultrasonic processor housing 105 of the imaging system 102. As shown in the embodiment of FIG. 2, the scanning assembly 108, the display 110, and the scanning processor 210 are separate components in communication with each other. However, in some embodiments, one or more of these components may be integrated (for example, the display and the scanning processor may be included in a single component).

In the example of FIG. 2, the scanning assembly 108 at least comprises an ultrasonic transducer 220 and a driving apparatus 240. The ultrasonic transducer 220 comprises a transducer array of transducer elements, such as a piezoelectric element converting electrical energy into ultrasonic waves and then detecting reflected ultrasonic waves.

The scanning assembly 108 may communicate with the scanning processor 210 to send raw scan data to an image processor. The scanning assembly 108 may optionally communicate with the display 110 so as to notify a user to reposition the scanning assembly as described above, or to receive information from the user (via user input 244).

In the example of FIG. 2, the scanning processor 210 includes an image processor 212, a memory 214, display output 216, and an ultrasonic engine 218. The ultrasonic engine 218 may drive activation of the transducer elements of the transducer 220, and in some embodiments, the driving apparatus 240 may be activated. Furthermore, the ultrasonic engine 218 may receive raw image data (for example, ultrasonic echoes) from the scanning assembly 108. The raw image data may be sent to the image processor 212 and/or a remote processor (for example, via a network) and be processed to form a displayable image of a tissue sample. It should be understood that in some embodiments, the image processor 212 may be included in the ultrasonic engine 218.

Information may be transmitted from the ultrasonic engine 218 and/or the image processor 212 to the user of the imaging system 102 via a display output 216 of the scanning processor 210. In one example, the user of the ultrasonic imaging system may include an ultrasonic technician, a nurse, or a physician such as a radiologist. For example, a processed image of scanned tissue may be sent to the display 110 via the display output 216. In another example, information related to parameters of the scanning (such as the progress of scanning) may be sent to the display 110 via the display output 216. The display 110 may include a user interface 242 configured to display images or other information to the user. Furthermore, the user interface 242 may be configured to receive an input from the user (such as by means of a user input unit 244), and send the input to the scanning processor 210. In one example, the user input unit 244 may be a touch screen of the display 110. However, other types of user input mechanisms are also possible, such as a mouse, a keyboard, and the like.

The scanning processor 210 may further include the memory 214. The storage 214 may include movable and/or permanent devices, and may include an optical memory, a semiconductor memory, and/or a magnetic memory. The storage 214 may include a volatile, non-volatile, dynamic, static, read/write, read only, random access, sequential access, and/or additional memory. The storage 214 may store non-transitory instructions executable by a controller or processor (such as a controller 218 or the image processor 212) so as to perform one or more methods or routines as described below. The storage 214 may store raw image data received from the scanning assembly 108, processed image data received from the image processor 212 or the remote processor, and/or additional information.

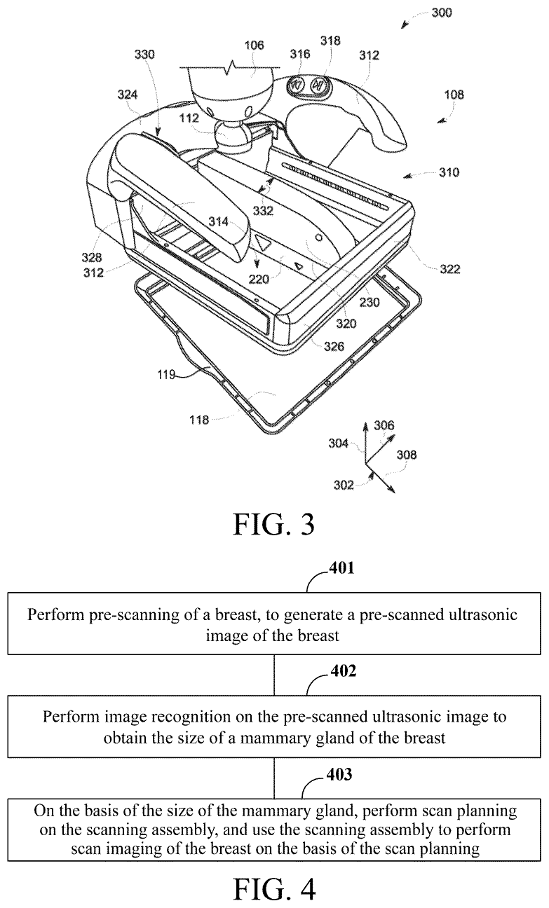

FIG. 3 shows a schematic diagram 300 of an isometric view of the scanning assembly 108 connected to the adjustable arm 106. The schematic diagram 300 includes a coordinate system 302, and the coordinate system 302 includes a vertical axis 304, a horizontal axis 306, and an abscissa axis 308.

The scanning assembly 108 includes a housing 310, the transducer module 220, and the module receiver 230. The housing 310 includes a frame 322 and a handle portion 324, and the handle portion includes two handles 312. The two handles 312 oppose each other across a transverse axis of the scanning assembly 108, and the transverse axis is centered on the adjustable arm 106 and defined relative to the transverse axis 308. The frame 322 is rectangular, and an inner periphery of the frame 322 defines an opening 314. The opening 314 provides space (e.g., a void volume) for translating the module receiver 230 and the transducer module 220 during a scanning process. In another example, the frame 322 can have another shape, such as a square having the square opening 314. In addition, the frame 322 has a thickness defined between an inner periphery and an outer periphery of the frame 322.

The frame 322 includes four sets of side walls (e.g., a set including inner and outer side walls, the inner side walls defining the opening 314). In particular, the frame 322 includes a front side wall 326 and a rear side wall 328, the rear side wall 328 is directly connected to the handle portion 324 of the housing 310, and the front side wall 326 is opposite to the rear side wall 328 with respect to the horizontal axis 306. The frame 322 further includes right and left side walls, the corresponding side walls opposing each other and both being in a plane defined by the vertical axis 304 and the transverse axis 308.

The frame 322 of the housing 310 further includes a top side and a bottom side, and the top side and the bottom side are defined relative to the vertical axis 304. The top side faces the adjustable arm 106. The film 118 is disposed across the opening 314. More specifically, the film 118 is connected to the bottom side of the frame 322. In an example, the film 118 is a diaphragm that remains tensioned across the opening 314. The film 118 may be made from a flexible but non-stretchable material, and the material is thin, waterproof, durable, highly acoustically transparent, resistant to chemical corrosion, and/or biocompatible. As described above, the bottom surface of the film 118 may contact a tissue (e.g., a breast) during scanning, and the upper surface of the film 118 may at least partially contact the transducer module 220 during scanning. As shown in FIG. 3, the film 118 is permanently connected to a hard-housing holding portion 119 surrounding the periphery of the film 118. The holding portion 119 is connected to the bottom side of the frame 322. In one example, the holding portion 119 can be fastened to a lip-like edge on the bottom side of the frame 322 of the housing 310, so that the film 118 does not become unconnected during scanning, but is still removably connected to the frame 322. The film 118 may not be permanently connected to the hard-housing holding portion 119, and thus the film 118 may be connected to the frame 322 without the hard-housing holding portion 119. Instead, the film 118 may be directly and removably connected to the frame 322.

The handle portion 324 of the housing 310 includes the two handles 312 for moving the scanning assembly 108 in space and positioning the scanning assembly 108 on a tissue (e.g., on the body of a patient). In an alternative embodiment, the housing 310 may not include the handle 312. In an example, the handle 312 may be integrally formed with the frame 322 of the housing 310. In another example, the handle 312 and the frame 322 may be formed separately and then mechanically connected together to form the entire housing 310 of the scanning assembly 108.

As shown in FIG. 3, the scanning assembly 108 is connected to the adjustable arm 106 by means of a ball joint 112 (e.g., a ball and socket connector). Specifically, a top dome portion of the handle portion 324 is connected to the ball joint 112. The top of the handle portion 324 includes a depression forming a socket, and a ball of the ball joint 112 is fit in the socket. The ball joint 112 is movable in multiple directions. For example, the ball joint 112 provides rotational motion of the scanning assembly relative to the adjustable arm 106. The ball joint 112 includes a locking mechanism for locking the ball joint 112 in place, thereby holding the scanning assembly 108 stationary relative to the adjustable arm 106. Furthermore, the ball joint 112 may also be configured to only rotate but not to move in multiple directions, such as oscillating.

Additionally, as shown in FIG. 3, the handle 312 of the handle portion 324 includes buttons for controlling scanning and adjusting the scanning assembly 108. Specifically, a first handle of the handles 312 includes a first weight adjustment button 316 and a second weight adjustment button 318. The first weight adjustment button 316 may reduce a load applied to the scanning assembly 108 from the adjustable arm 106. The second weight adjustment button 318 may increase a load applied to the scanning assembly 108 from the adjustable arm 106. Increasing the load applied to the scanning assembly 108 may increase the pressure and the amount of pressing applied to the tissue on which the scanning assembly 108 is placed. Furthermore, increasing the load applied to the scanning assembly increases the effective weight of the scanning assembly on the tissue to be scanned. In one example, increasing the load may press a tissue of a patient, such as a breast. In this way, varying amounts of pressure (e.g., load) may be applied consistently with the scanning assembly 108 during scanning, so as to obtain high quality images by using the transducer module 220.

Before the scanning process, a user (e.g., an ultrasonic technician or physician) may position the scanning assembly 108 on a patient or a tissue. Once the scanning assembly 108 is properly positioned, the user may adjust a weight (e.g., adjust an amount of pressing) of the scanning assembly 108 on the patient by means of the first weight adjustment button 316 and/or the second weight adjustment button 318. Then, the user may initiate the scanning process by means of additional control on the handle portion 324 of the housing 310. For example, as shown in FIG. 3, the second handle of the handles 312 includes two additional buttons 330 (not shown separately). The two additional buttons 330 may include a first button for initiating a scan (e.g., once the scanning assembly has been placed on the tissue/patient and an amount of pressing has been selected) and a second button for stopping the scan. In one example, once the first button is selected, the ball joint 112 may be locked, thereby stopping transverse and horizontal movement of the scanning assembly 108.

The module receiver 230 is positioned within the housing 310. Specifically, the module receiver 230 is mechanically connected to a first end of the housing 310 at a rear side wall 328 of the frame 322, and the first end is closer to the adjustable arm 106 than a second end of the housing 310. The second end of the housing 310 is located at a front side wall 326 of the frame 322. In one example, the module receiver 230 is connected to the first end by means of a protruding portion of the module receiver 230, the protruding portion is connected to the motor 230, and the protruding portion is connected to the motor of the module receiver 230.

As described above, the housing 310 is configured to remain stationary during scanning. In other words, once the weight applied to the scanning assembly 108 is adjusted by means of the adjustable arm 106 and then the ball joint 112 is locked, the housing 310 may remain in the resting position without translating in the horizontal or transverse direction. However, the housing 310 may still translate vertically as the adjustable arm 106 move vertically.

Instead, the module receiver 230 is configured to translate relative to the housing 310 during scanning. As shown in FIG. 3, the module receiver 230 translates horizontally along a horizontal axis 306 relative to the housing 310. The motor of the module receiver 230 may slide the module receiver 230 along an upper surface of the first end of the housing 310.

The transducer module 220 is removably connected to the module receiver 230. Therefore, during scanning, the transducer module 220 and the module receiver 230 translate horizontally. During scanning, the transducer module 220 sweeps horizontally across the breast under the control of the motor of the module receiver 230, and at the same time, a contact surface of the transducer module 220 contacts the film 118. The transducer module 220 and the module receiver 230 are connected together at a module interface 320. The module receiver 230 has a width 332 that is the same as a width of the transducer module 220. In an alternative embodiment, the width 332 of the module receiver may be different from the width of the transducer module 220. In some embodiments, the module interface 320 includes a connector between the transducer module 220 and the module receiver 230, and the connector includes mechanical and electrical connections.

The embodiments of the present application provide a method for performing ultrasonic scanning on a breast. The ultrasonic scanning is at least partially performed by a scanning assembly. The scanning assembly is, for example, the scanning assembly 108 of the ultrasonic imaging system 102 shown in FIG. 1, and includes a frame 104. A scanning probe and a driving apparatus are accommodated in the frame 104, and the driving apparatus drives the scanning probe to move within the frame to perform ultrasonic scanning.

FIG. 4 is a schematic diagram of a method for performing ultrasonic scanning on a breast according to an embodiment of the present application. As shown in FIG. 4, the method comprises:

401: Performing pre-scanning of the breast, to generate a pre-scanned ultrasonic image of the breast;

402: Performing image recognition on the pre-scanned ultrasonic image, to obtain the size of a mammary gland of the breast; and

403: On the basis of the size of the mammary gland, performing scan planning on the scanning assembly. and using the scanning assembly to perform scan imaging of the breast on the basis of the scan planning.

It should be noted that FIG. 4 merely schematically illustrates the embodiments of the present application, but the present application is not limited thereto. For example, the order of execution between operations may be appropriately adjusted. In addition, some other operations may be added or some operations may be omitted. Those skilled in the art may make appropriate variations according to the above content, rather than being limited to the above disclosure of FIG. 4.

According to the described embodiments, pre-scanning is performed before a main scan to determine the size of a mammary gland of a breast, scan planning is performed according to the size of the mammary gland of the breast, and the main scan is performed on the basis of the scan planning. Thus, it is possible to save scanning time and improve a scanning success rate by means of performing scan planning in a targeted manner, and not indiscriminately using a fixed number of scans with a fixed position.

In operation 401, pre-scanning the breast may be, for example, acquiring ultrasonic data about the breast in at least one angular direction, the ultrasonic data including at least ultrasonic data of a nipple edge of the breast and ultrasonic data extending outward along the nipple edge to an edge of the mammary gland. Thus, a pre-scanned ultrasonic image containing the complete mammary gland can be obtained, and the size of the mammary gland of the breast can be determined on the basis of the pre-scanned ultrasonic image.

In the above embodiments, a probe used for performing pre-scanning may be an additional probe or the scanning probe (i.e., the probe accommodated in the scanning assembly). The additional probe may be, for example, a two-dimensional (2D) probe, a volumetric ultrasonic probe, or another probe. The volumetric ultrasonic probe may be, for example, a three-dimensional (3D) probe, a four-dimensional (4D) probe, or the like.

In one example, the additional probe is another probe, different from the scanning probe, of the ultrasonic imaging system, which is attached to the ultrasonic imaging system by means of a fixed connection or a pluggable connection (e.g., by a probe connector). Compared with the scanning probe provided within the scanning assembly and automatically driven, the above additional probe can provide a higher degree of flexibility for the user, and is suitable for evaluating the breast quickly. Of course, in a further example, the additional probe may also be a probe different from that of the ultrasonic imaging system, e.g. a handheld probe or a probe of another ultrasonic device. The evaluation results may be displayed to the user or transmitted to the ultrasonic imaging system at the same time.

In other examples, the probe used for pre-scanning may also be the scanning probe itself. The user may control the positioning of the scanning probe on the surface of the breast by operating an ultrasonic imaging system (such as the ultrasonic imaging system 102). At this time, the operator may not drive the movement of the scanning probe, but rather only perform imaging in a normal imaging mode, thereby evaluating the breast quickly. This example provides a lower-cost option for the operator since no extra additional device (such as an additional probe) is required.

In the above embodiments, the at least one angular direction includes a plurality of angular directions, and the plurality of angular directions may be uniformly distributed in a circumferential direction.

Taking as an example the at least one angular direction including four angular directions, the four angular directions may be four directions of 0 degrees, 90 degrees, 180 degrees, and 270 degrees, which are centered on the nipple.

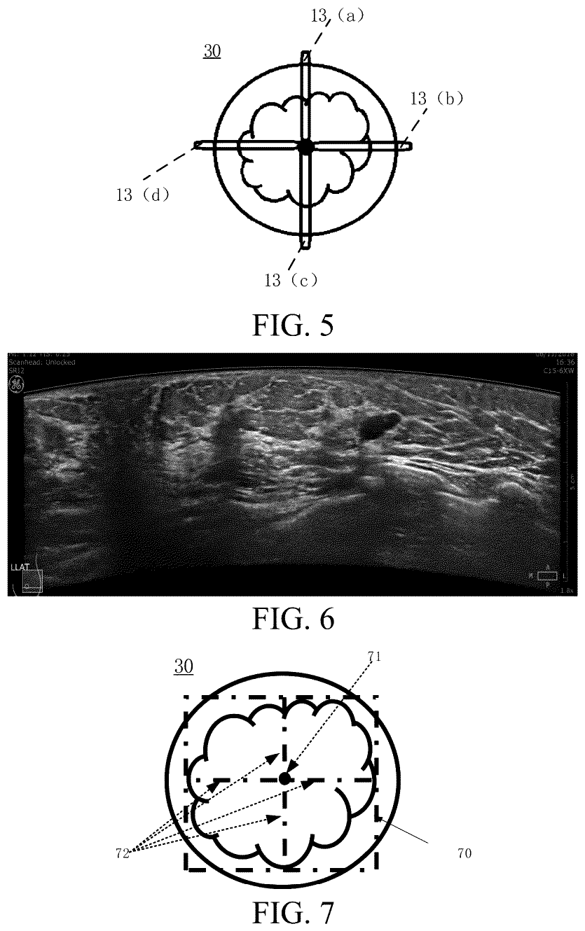

FIG. 5 is a schematic view of pre-scanning the breast 30 in the above four angular directions, showing the scanning probe in four positions, i.e., 13(a), 13(b), 13(c), and 13(d), in the above four angular directions. FIG. 6 is a schematic diagram of a pre-scanned ultrasonic image obtained after pre-scanning the breast 30 in any direction shown in FIG. 5.

As shown in FIG. 5, the operator may align the edge of the scanning probe with the nipple and then place the scanning probe in the four positions in sequence, performing one scan at each position to obtain four pre-scanned ultrasonic images, as shown in FIG. 6.

For example, the scanning probe performs one scan at position 13(a) to obtain a pre-scanned ultrasonic image; then, performs one scan at position 13(b) to obtain a pre-scanned ultrasonic image; then, performs one scan at position 13(c) to obtain a pre-scanned ultrasonic image; and finally, performs one scan at position 13(d) to obtain a pre-scanned ultrasonic image. Thus, it can be ensured that representative pre-scanned ultrasonic images are obtained on the basis of as few pre-scans as possible, providing a basis for the subsequent determination of the size of the mammary gland.

The above description has been made using four angular directions as an example, and the present application is not limited thereto. In some embodiments, pre-scanning may also be performed in another number of angular directions to obtain corresponding pre-scanned ultrasonic images. For example, it is also possible that pre-scanning is performed in each of three angular directions, with the angle between the respective angular directions being 120 degrees, to obtain three pre-scanned ultrasonic images, and the size of the mammary gland is determined according to the three pre-scanned ultrasonic images. It is also possible that pre-scanning is performed in each of two angular directions, with the angle between the respective angular directions being 180 degrees, to obtain two pre-scanned ultrasonic images, and the size of the mammary gland is determined according to the two pre-scanned ultrasonic images. In an extreme example, it is also possible that pre-scanning is performed in only one angular direction to obtain one pre-scanned ultrasonic image, for example, pre-scanned ultrasonic images at other angles being inferred according to the characteristics of breast symmetry, and the size of the mammary gland being determined from the obtained pre-scanned ultrasonic image and the inferred pre-scanned ultrasonic images.

In operation 402, image recognition is performed on the pre-scanned ultrasonic image, which may be, for example, recognizing the mammary gland in the pre-scanned ultrasonic image, determining an outer edge of the mammary gland, and obtaining the size of the mammary gland of the breast on the basis of the outer edge of the mammary gland.

In the above embodiment, a specific method of image recognition is not limited.

In one possible example, the image recognition method may be defined by one or more algorithms to recognize a section of an object of interest to be scanned (e.g., a breast) on the basis of one or more anatomical features (e.g., boundary, thickness, pixel value change, edge or inner layer, etc.) within the pre-scanned ultrasonic image, a modality or pattern (e.g., color blood flow) of the pre-scanned ultrasonic image, etc. The one or more anatomical features may represent a feature of pixels and/or voxels of the pre-scanned ultrasonic image, such as a histogram of oriented gradients, a point feature, a covariance feature, a binary mode feature, and the like. For example, the image recognition method may be defined using prediction of object recognition within the pre-scanned ultrasonic image using one or more deep neural networks.

In another possible example, the image recognition method may correspond to an artificial neural network formed by a controller circuit and/or a remote server. The image recognition method may be divided into two or more layers, such as an input layer for receiving an input image (e.g., a pre-scanned ultrasonic image), an output layer for outputting an output image, and/or one or more intermediate layers. Layers of the neural network represent different groups or sets of artificial neurons, and may represent different functions that are executed by the controller circuit with respect to the input image (e.g., a pre-scanned ultrasonic image) to recognize an object of the input image and determine a section of an anatomical structure of interest shown in the input image. An artificial neuron in a layer of the neural network may examine an individual pixel in the input image. The artificial neurons use different weights in a function applied to the input image, so as to attempt to identify an object in the input image. The neural network produces an output image by assigning or associating different pixels in the output image with different anatomical features on the basis of the analysis of pixel characteristics.

In another possible example, the image recognition method is defined by a plurality of training images, and the plurality of training images may be grouped into different anatomical planes of interest of the anatomical structure of interest. The training images may represent different orientations and/or cross sections of the anatomical structure of interest corresponding to different fields of view. Additionally or alternatively, the image recognition method may be defined by the controller circuit on the basis of a classification model. The classification model may correspond to a machine learning algorithm based on a classifier (e.g., a random forest classifier, principal component analysis, etc.) configured to identify and/or assign anatomical features to multiple types or categories based on overall shape, spatial position relative to the anatomical structure of interest, intensity, etc.

In the above embodiment, still taking pre-scanning being performed in each of the four angular directions shown in FIG. 5 as an example, according to an embodiment of the present application, an outer edge 70 of a mammary gland shown in FIG. 7 is obtained by performing image recognition on the obtained pre-scanned ultrasonic image shown in FIG. 6 by using any of the image recognition methods exemplified in the above example, and the size of the mammary gland is obtained on the basis of the outer edge 70 of the mammary gland. In the example of FIG. 7, the position 71 of the nipple and four positions 72 of the scanning probe are also shown.

In the above embodiment, by means of performing image recognition on the pre-scanned ultrasonic image, a chest wall in the pre-scanned ultrasonic image may also be obtained, and the depth of scan imaging may be determined on the basis of the chest wall.

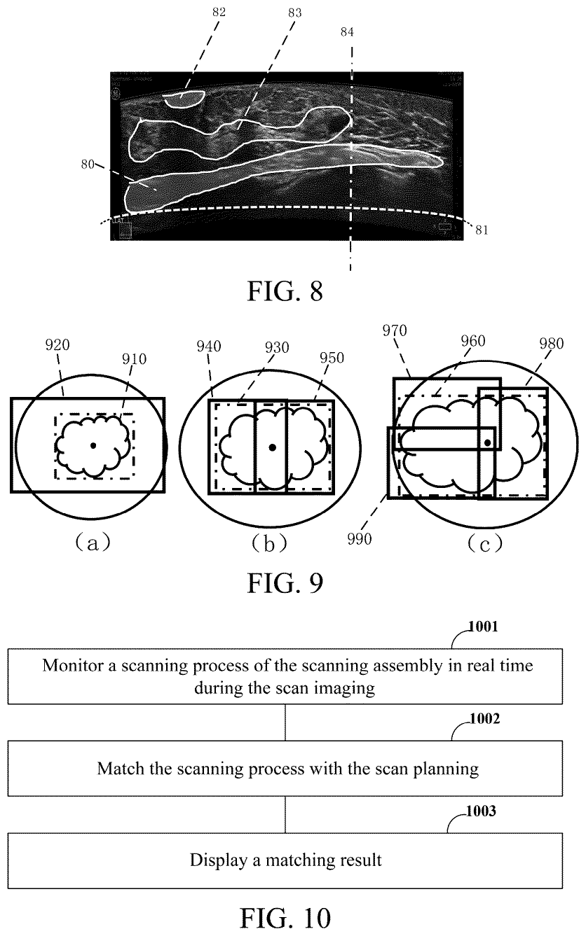

Still taking pre-scanning being performed in each of the four angular directions shown in FIG. 5 as an example, after obtaining the four pre-scanned ultrasonic images, by means of performing image recognition on the four pre-scanned ultrasonic images, not only is the outer edge 70 of the mammary gland shown in FIG. 7 obtained, but the chest wall 80 shown in FIG. 8 is also obtained. The size of the mammary gland can be obtained on the basis of the outer edge 70 of the mammary gland shown in FIG. 7, and the depth of scan imaging can be obtained on the basis of the chest wall 80 shown in FIG. 8.

In the above embodiment, as shown in FIG. 8, the depth of scan imaging may be a maximum value 81 among depths of the chest wall 80 in the pre-scanned ultrasonic image. Thus, the integrity of the scanned image can be ensured, and the mammary gland and the deepest chest wall can be completely presented in the image. At the same time, too many images below the chest wall are not introduced, so that the display effect of the image is better. Specifically, in conventional automated breast ultrasound screening, the depth value needs to be set in advance. If the depth value is too high, clinically meaningful images (e.g., images above the chest wall) account for too low a proportion of the entire image and are too small in size, which is disadvantageous for a physician to observe the images. If the depth value is too low, valuable anatomical features in the images may be lost. By comparison between the two, the user may be more inclined to ensure the integrity of the anatomical feature, so a larger depth value is selected. This will degrade image quality. In the above embodiments of the present application, by simultaneously recognizing anatomical features of the mammary gland and the chest wall, valuable anatomical feature size information in both the depth direction and the circumferential direction can be accurately provided without requiring an additional ultrasound screening step. In this way, the image quality can be improved while ensuring integrity, without requiring an additional scanning step.

In the above embodiments, as shown in FIG. 8, image recognition is performed on the anatomical features of the pre-scanned ultrasonic image to obtain the anatomical features of the nipple 82, the mammary gland 83 and the chest wall 80, and the optimum depth of scan imaging, i.e., the maximum value 81 among the depths of the chest wall 80, and the optimum circumferential range of scan imaging, i.e., the outer edge 84 of the mammary gland 83, can be accurately determined. Thus, valuable anatomical feature size information in the depth direction and in the circumferential direction can be determined without an additional ultrasonic screening step and pre-setting of depth values, and scanning is performed on the basis of the obtained anatomical feature size information, so that image quality can be improved while ensuring the integrity of scan imaging, and no additional scanning steps are required.

In operation 403, scan planning may include at least one of the number of scans, a scanning position, and a scanning stroke of the scanning assembly. The number of scans refers to, for example, the number of times required for the scanning assembly to complete the scanning of the breast. The scanning position refers to, for example, a placement position of the scanning assembly during each scan. The scanning stroke refers to, for example, a path in which the scanning assembly moves during each scan.

Thus, scan planning is performed according to the size of the mammary gland, and the breast is scanned a reasonable number of times in a targeted manner, which can save scanning time and improve a scanning success rate.

In one possible example, in single-scan imaging, scan planning may be: planning so that the driving apparatus does not drive the scanning probe to move outside the range of the mammary gland. Thus, scanning of a region outside the mammary gland can be avoided, thereby saving scanning time and improving scanning efficiency.

In another possible example, in multi-scan imaging, scan planning may be: planning so that the driving apparatus does not drive the scanning probe to scan an overlapping region between a plurality of scans. Thus, repeated scanning can be avoided, saving scanning time and improving scanning efficiency.

FIG. 9 is a schematic diagram of scan planning corresponding to different sizes of mammary glands. As shown in FIG. 9, in (a), the mammary gland size 910 is relatively small, and scan planning is a single scan; that is, only one scan is performed in the range of a frame 920. In (b), the mammary gland size 930 is relatively large compared with (a), and scan planning is two scans; that is, one scan is performed in each of blocks 940 and 950. Optionally, for an overlapping region of blocks 940 and 950, the driving apparatus may drive the scanning probe not to scan in the overlapping region according to scan planning. In (c), the mammary gland size 960 is relatively the largest compared with (a) and (b), and scan planning is three scans; that is, one scan is performed in each of blocks 970, 980 and 990. Optionally, for overlapping regions of blocks 970, 980 and 990, the driving apparatus may drive the scanning probe not to scan in the overlapping regions according to scan planning.

According to the above embodiment, scan imaging of the breast is performed according to scan planning, so that the scanning time is shortened, and the scanning success rate is improved.

In some embodiments, it is further possible to match the scanning process and scan planning during scan imaging and display the matching result, so that the operator scans with reference to the matching of the scanning process and scan planning.

FIG. 10 is a schematic diagram of matching a scanning process and scan planning. As shown in FIG. 10, the method includes:

1001: Monitoring a scanning process of a scanning assembly in real time during scan imaging;

1002: Matching the scanning process and the scan planning; and

1003: Displaying a matching result.

In the above example, the monitoring method is not limited. For example, a sensor may be installed on the scanning assembly to perform real-time monitoring, so as to determine orientation information such as a position and a tilt angle of the scanning assembly, or the orientation information of the scanning assembly may be generally determined according to an image recognition result, or the two methods may be combined. The real-time monitoring method depends on specific requirements, and the present application is not limited thereto.

According to the above embodiment, the degree of matching between the scanning process of the scanning assembly and the scan planning may be monitored in real time, and when a scanning deviation occurs, it can be corrected quickly, thereby improving the accuracy rate of scanning.

In some embodiments, scan planning may also be displayed on a display.

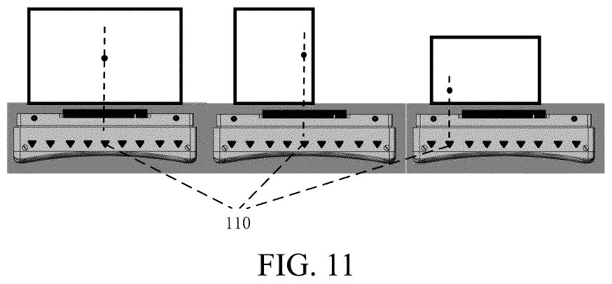

FIG. 11 is a schematic diagram of displaying scan planning on a display. In this example, still taking the scan planning shown in FIG. 9 as an example, as shown in FIG. 11, a plurality of LED lights 110 are provided on the scanning probe to indicate a nipple position. Corresponding to different nipple alignment positions, different LED lights 110 light up to prompt the operator to align the nipple at the position where the LED light 110 lights up to start scanning.

The above is merely an example, and the number and implementation of the LED lights are not limited in the present application.

According to the above embodiments, scanning accuracy and the success rate of scanning are improved.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

As can be seen from the above embodiments, the size of the mammary gland is determined by pre-scanning the breast before a main scan, scan planning is performed on the scanning assembly on the basis of the size of the mammary gland, and scan imaging of the breast is performed on the basis of the scan planning. This can save scanning time and improve a scanning success rate.

The embodiments of the present application further provide an ultrasonic imaging system. The ultrasonic imaging system includes a scanning assembly and a processor, the scanning assembly being used so move over the surface of a breast so as to acquire ultrasonic echo signals. The specific implementation of the scanning assembly has been described above and will not be described again here.

In the above embodiment, the processor is configured to execute the method of the foregoing embodiments, and since the specific implementation of the method has been described in the foregoing embodiments, the contents of which are incorporated herein, no further description is provided here.

The embodiments of the present application further provide a non-transitory computer-readable medium, having a computer program stored thereon, the computer program having at least one code segment, and the at least one code segment being executable by a machine, so that the machine executes steps of the method according to the foregoing embodiments. Since the specific implementation of the method has been described in the foregoing embodiments, the contents of which are incorporated herein, no further description is provided here.

The above method of the present application may be implemented by hardware, or may be implemented by hardware in combination with software. The present application relates to such a computer-readable program that, when executed by a logic component, the program enables the logic component to implement the constituent components described above, or enables the logic component to implement various methods or steps as described above. The present application further relates to a storage medium for storing the above program, such as a hard disk, a disk, an optical disk, a DVD, a flash memory, etc.

The method described with reference to the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A method for performing ultrasonic scanning on a breast, the method being at least partially performed by a scanning assembly, the scanning assembly comprising a frame, a scanning probe and a driving apparatus being disposed in the frame, and the driving apparatus driving the scanning probe to move within the frame to perform the ultrasonic scanning, and the method comprising:
pre-scanning the breast to generate a pre-scanned ultrasonic image of the breast;
performing image recognition on the pre-scanned ultrasonic image, to obtain the size of a mammary gland of the breast; and
performing scan planning on the scanning assembly based on the size of the mammary gland, and
performing scan imaging of the breast, based on the scan planning, using the scanning assembly.

2. The method according to claim 1, wherein pre-scanning of the breast comprises:
acquiring ultrasonic data about the breast in at least one angular direction, the ultrasonic data comprising at least ultrasonic data of a nipple edge of the breast and ultrasonic data extending outward along the nipple edge to an edge of the mammary gland.

3. The method according to claim 1, wherein,
a probe used for performing the pre-scanning is an additional probe or the scanning probe, wherein the additional probe comprises at least one of a 2D probe and a volumetric ultrasonic probe.

4. The method according to claim 2, wherein the at least one angular direction comprises a plurality of angular directions, the plurality of angular directions being uniformly distributed in a circumferential direction.

5. The method according to claim 1, wherein performing image recognition on the pre-scanned ultrasonic image to obtain the size of the mammary gland of the breast comprises:
recognizing the mammary gland in the pre-scanned ultrasonic image, determining an outer edge of the mammary gland, and obtaining the size of the mammary gland of the breast on the basis of the outer edge of the mammary gland.

6. The method according to claim 1, wherein the method further comprises:
performing image recognition on the pre-scanned ultrasonic image to obtain a chest wall in the pre-scanned ultrasonic image, and determining a depth of the scan imaging on the basis of the chest wall.

7. The method according to claim 6, wherein,
the depth of the scan imaging is a maximum value among chest wall depths in the pre-scanned ultrasonic image.

8. The method according to claim 1, wherein the scan planning comprises at least one of a number of scans, a scanning position, and a scanning stroke of the scanning assembly.

9. The method according to claim 1, wherein the method further comprises:
displaying the scan planning on a display.

10. The method according to claim 1, wherein the method further comprises:
monitoring a scanning process of the scanning assembly in real time during the scan imaging;
matching the scanning process and the scan planning; and
displaying a matching result.

11. The method according to claim 1, wherein the scan planning comprises:
in single-scan imaging, planning so that the driving apparatus does not drive the scanning probe to move outside the mammary gland; and/or
in multi-scan imaging, planning so that the driving apparatus does not drive the scanning probe to scan an overlapping region between a plurality of scans.

12. An ultrasonic imaging system, the ultrasonic imaging system comprising:
a scanning assembly moving over the surface of a breast to acquire ultrasonic echo signals;
a memory storing instructions; and
a processor configured to execute the instructions to control the ultrasonic imagining system to:
pre-scan the breast to generate a pre-scanned ultrasonic image of the breast;
perform image recognition on the pre-scanned ultrasonic image, to obtain the size of a mammary gland of the breast; and
perform scan planning on the scanning assembly based on the size of the mammary gland, and
perform scan imaging of the breast based on the scan planning.

13. The ultrasonic imaging system according to claim 12, wherein pre-scanning of the breast comprises:
acquiring ultrasonic data about the breast in at least one angular direction, the ultrasonic data comprising at least ultrasonic data of a nipple edge of the breast and ultrasonic data extending outward along the nipple edge to an edge of the mammary gland.

14. The ultrasonic imaging system according to claim 12,
wherein the scan imaging of the breast is performed by a scanning probe, and
wherein the pre-scanning is performed on one of an additional probe and the scanning probe, wherein the additional probe comprises at least one of a 2D probe and a volumetric ultrasonic probe.

15. The ultrasonic imaging system according to claim 13, wherein the at least one angular direction comprises a plurality of angular directions, the plurality of angular directions being uniformly distributed in a circumferential direction.

16. The ultrasonic imaging system according to claim 12, wherein performing image recognition on the pre-scanned ultrasonic image to obtain the size of the mammary gland of the breast comprises:
recognizing the mammary gland in the pre-scanned ultrasonic image, determining an outer edge of the mammary gland, and obtaining the size of the mammary gland of the breast on the basis of the outer edge of the mammary gland.

17. The ultrasonic imaging system according to claim 12, wherein the processor is configured to execute the instructions to control the ultrasonic imagining system to perform image recognition on the pre-scanned ultrasonic image to obtain a chest wall in the pre-scanned ultrasonic image, and determine a depth of the scan imaging on the basis of the chest wall.

18. The ultrasonic imaging system according to claim 17, wherein,
the depth of the scan imaging is a maximum value among chest wall depths in the pre-scanned ultrasonic image.

19. The method according to claim 12, wherein the scan planning comprises at least one of a number of scans, a scanning position, and a scanning stroke of the scanning assembly.

20. A non-transitory computer-readable medium, the non-transitory computer-readable medium having a computer program stored thereon, the computer program having at least one code segment, and the at least one code segment being executable by a machine, so that the machine executes steps of:
pre-scanning the breast to generate a pre-scanned ultrasonic image of the breast;
performing image recognition on the pre-scanned ultrasonic image, to obtain the size of a mammary gland of the breast; and
performing scan planning on the scanning assembly based on the size of the mammary gland, and
performing scan imaging of the breast based on the scan planning.
